(19) United States
(12) Patent Application Publication
(10) Pub. No.: US 2021/0196621 A1
(43) Pub. Date: Jul. 1, 2021

(54) ALCOHOL-FREE BOTANICAL ORAL CARE PRODUCTS

(71) Applicant: The Trustees of Columbia University in the City of New York, New York, NY (US)

(72) Inventors: Shanta M. Modak, River Edge, NJ (US); Chathuranga C. De Silva, Cliffside Park, NJ (US)

(21) Appl. No.: 17/057,891

(22) PCT Filed: May 23, 2019
(86) PCT No.: PCT/US19/33799

§ 371 Date: Nov. 23, 2020

Related U.S. Application Data
(60) Provisional application No. 62/675,514, filed on May 23, 2018.

(51) Int. Cl. A61K 8/9789; A61K 8/34; A61K 8/60; A61K 8/37; A61K 8/368; A61K 8/19; A61K 8/43; A61K 36/752; A61K 36/185; A61K 31/045; A61K 31/05; A61K 47/26; A61K 47/44; A61K 47/10; A61K 8/9794; A61K 8/365; A61K 8/04; A61Q 11/00

(52) U.S. Cl. CPC A61K 8/9789; A61K 2800/49; A61K 8/345; A61K 8/602; A61K 8/375; A61K 8/368; A61K 8/19; A61K 8/43; A61K 36/752; A61K 36/185; A61K 31/045; A61K 31/05; A61K 47/26; A61K 47/44; A61K 47/10; A61K 8/9794; A61K 8/365; A61K 8/046; A61Q 11/00; A61K 2800/5922; A61K 8/34

(57) ABSTRACT

The present technology relates to alcohol-free botanical oral care compositions that are stable, natural, non-irritating and effective against oral pathogens. In various embodiments, these compositions can be in the form of an oral rinse, a toothpaste, an oral patch, an oral spray, a dental floss, or a lozenge. The present technology also relates to methods of treating a wound, and methods of coating a medical device, using any of the compounds discussed herein.

### BACKGROUND

The present technology relates to alcohol-free botanical oral care compositions that are stable, natural, non-irritating and effective against oral pathogens.

The oral cavity and teeth, with its deep periodontal pockets, is an ideal reservoir for microbial species. Oral micro-organisms spread easily into the blood stream through inflamed gingival tissue, deep caries cavities, or ulcers of the mucosa. Bacteremia is generally not a threat to healthy persons, but it may be harmful, and even fatal, to those with weakened general health.

The fungus Candida albicans and other Candida species are common organisms of the human oral flora. Studies have shown that the oral prevalence of these organisms in healthy adult humans ranges from 3 to 47%. While many factors predispose the host to the development of oral candidiasis, old age, debility and denture wearing are major causes of oral candidiasis.

Various antibacterial agents have been incorporated in oral care formulations to address this issue. However, many antibacterial agents negatively interact with one or more other components in the same formulation, especially surfactants and solubilizers used in these oral care formulations. This can diminish the effective performance of such oral care compositions. There is a continuing interest to develop oral care compositions which improve the efficacy and/or bioavailability of actives for combating plaque, gingivitis, periodontitis or diseases of the oral cavity, as well as producing anti-inflammatory effects.

It has been reported that routine use of synthetic antimicrobials may be harmful to human health and environment. The recent trend in the use of environment-friendly products has led to the increased use of natural products to treat diseases. The antiseptic properties of natural products such as certain aromatic and medicinal plants and their extracts have been investigated. The essential oils from certain plants have been characterized and found to possess antimicrobial, antiviral, and anti-inflammatory properties.

The Centers for Disease Control and Prevention (CDC) have issued guidelines recommending the use of alcohol-based products (60-70% alcohol) for skin disinfection to reduce the transmission of pathogenic organisms to patients and personnel in health care settings. However, alcohol-based hand sanitizers, although effective in rapidly killing germs, disrupt the skin's moisture and pH balance by stripping away the natural oils, leaving the skin dry, cracked, and more susceptible to infections. Further, alcohol-based hand sanitizers with 60-70% alcohol are highly flammable and can be misused with toxic consequences. According to a 2007 report published by the American Association of Poison Control Center, in 2006 there were approximately 12000 incidents of alcohol-poisoning directly attributed to alcohol-based hand sanitizers.

To make use of the beneficial effect of essential oils with reduced risk of deleterious effects, low concentrations of various essential oils, plant fruit extracts, and botanical anti-inflammatory or anti-irritant agents in combination, have been evaluated for their synergistic antimicrobial efficacy. Oral care compositions that contain natural or botanical-based active ingredients are desirable.

It has been well documented that oral hygiene is extremely important not only to prevent dental plaque, gingivitis and bad breath, but also to prevent ventilator associated pneumoniae (VAP) and other infections resulting from oral pathogens invading the body in compromised patients. It has been shown in the literature that prophylactic use of 0.12% chlorhexidine containing mouth rinse was found to reduce the risk of VAP. An alcohol-free botanical oral rinse (BOR), as well as other oral care products, have herein been successfully developed.

International Publication No. WO 2014/092999 A1 (Modak et al.) discloses oral rinses prepared with botanical blend comprising thymol, vanilla extract and PHMB (or chlorhexidine). In antimicrobial assays, PHMB showed synergistic activity with thymol and vanilla extract while chlorhexidine did not.

U.S. Application Publication No. 2016/0374352 (Modak et al.) discloses botanical oral rinses comprising thymol or menthol or a combination thereof which were combined with grapefruit seed extract, propanediol (for example, that available under the trade name Zemea® from DuPont Tate & Lyle BioProducts of Loudon, Tenn., USA) and pomegranate extract, with and without chlorhexidine and evaluated their efficacy. It was found that pomegranate extract significantly enhanced the antimicrobial efficacy, and that chlorhexidine further synergistically enhanced the antibacterial activity of blends containing pomegranate extract. Chlorhexidine did not show significant synergism with the botanical blend absent pomegranate extract.

An ongoing need exists for low alcohol or alcohol-free, natural compositions that are non-irritating, safe, and effective for repeated use in oral care products.

### BRIEF SUMMARY

In certain embodiments, the present technology is directed to an alcohol-free botanical oral care composition that achieves 3 or more logio reduction from control bacterial counts within 20 seconds of contact, comprising:

(a) a combination of 0.1 to 0.5% w/w pomegranate extract/oil and 0.3 to 1% w/w grapefruit seed extract;

(b) 0.05 to 0.08% w/w thymol, or 0.05 to 0.08% w/w menthol, or a combination thereof;

(c) 2 to 5% w/w 1,3 propanediol, or 5 to 15% w/w glycerin, or a combination thereof;

(d) 0.03 to 0.06% w/w peppermint oil, or 0.03 to 0.06% w/w spearmint oil, or a combination thereof;

(e) 0.3 to 0.8% w/w caprylyl capryl glucoside solubilizer; and

(f) 0.3 to 0.8% w/w one or more of organic surfactants selected from the group consisting of sorbitan mono laurate (e.g., Tween® 20), sorbitan mono oleate (e.g., Tween® 80), sorbitan oleate decylglucoside, sorbitan sugar (e.g., Poly Suga® mulse);
wherein the ratio of solubilizer to surfactant ranges from 1:1 to 1:20 and the combined concentration of solubilizer and surfactant does not exceed 2% of the composition.

In certain embodiments, any composition herein achieves 3 or more logio reduction from control bacterial counts within 30 seconds of contact.

In certain embodiments, the oral care composition of the technology further comprises 0.1 to 0.2% w/w benzoic acid, or 0.1 to 0.2% w/w sodium benzoate, or a combination thereof

In certain embodiments, the compositions therein further comprise one or more of anti-irritant or anti-inflammatory agents. These can include, e.g., calendula extract, zinc salicylate, methyl salicylate, and aloe gel extract.

In certain embodiments, the oral care composition of the technology further comprises xylitol, erythritol, sorbitol, or a combination thereof.

In certain embodiments, the oral care composition of the technology further comprises chlorhexidine gluconate.

In certain embodiments, any of the oral care compositions discussed herein can further comprise a natural colorant. In certain embodiments, the natural colorant is a copper salt, chlorophyllin, a botanical (e.g., a botanical extract) or a combination thereof. In other embodiments, any of the oral care compositions discussed herein can be substantially free of a colorant—i.e., contains less than 0.1% of a colorant.

In certain embodiments, an oral care composition of the present technology is part of a consumer product in the form of an oral rinse, a toothpaste, an oral patch, a dental floss, or a lozenge.

In certain embodiments, an oral care composition of the present technology further comprises a mixture of: (a) a hydrophilic polymer; and (b) a hydrophobic-hydrophilic polymer.

In certain embodiments, the present technology is directed to methods of treating a wound comprising contacting the wound with any of the compositions discussed herein, and medical devices coated with any of the compositions discussed herein.

In certain embodiments, the present technology is directed to an alcohol-free natural toothpaste composition that achieves more than 15 mm zone of inhibition against S. aureus comprising:

(a) a combination of 0.1 to 1% w/w pomegranate extract/oil and 0.5 to 2% w/w grapefruit seed extract;

(b) 0.05 to 1% w/w thymol, or 0.05 to 1% w/w menthol, or a combination thereof;

(c) 2 to 20% w/w 1,3 propanediol, or 8 to 50% w/w glycerin, or a combination thereof;

(d) 0.03 to 1% w/w peppermint oil, or 0.03 to 1% w/w spearmint oil, or a combination thereof.

In certain embodiments, the compositions can further comprise any of the following:

(e) 0.2 to 1% w/w caprylyl capryl glucoside solubilizer; and

(f) 0.2 to 1% w/w one or more organic surfactants selected from the group consisting of sorbitan mono laurate, sorbitan mono oleate, cocoamidopropyl betaine, bio-Saponin, sodium cocoyl glutamate, and sorbitan oleate decylglucoside.

In certain embodiments, the compositions herein can contain any of the following additional ingredients:

0.05 to 1% w/w eucalyptol, or 0.01 to 2% w/w benzyl alcohol, or a combination thereof; 0.1 to 45% w/w calcium carbonate, or 0.1 to 40% w/w sodium bicarbonate, or optionally up to 20% w/w texturing agents, or combination thereof;

0.01 to 5% w/w carrageenan, or 0.01 to 1% w/w methyl cellulose, or 0.1 to 2% w/w xanthan gum, or 0.5 to 20% w/w coconut oil thickeners, or combinations thereof.

In certain embodiments, a toothpaste composition of the present technology further comprises 0.1 to 0.5% w/w benzoic acid, or 0.1 to 0.5% w/w sodium benzoate, or a combination thereof.

In certain embodiments, a composition of the present technology further comprises one or more of: an anti-irritant or anti-inflammatory agent, for example, calendula extract, methyl salicylate, zinc salicylate, or aloe gel extract; or one or more of xylitol, erythritol, sorbitol or a combination thereof In certain embodiments, a composition of the present technology further comprises one or more of the following flavoring agents: clove extract or powder, clove bud oil, cardamom extract or powder, peppermint flavor or extract, spearmint flavor or extract, licorice root extract or powder, white oak bark extract or powder, gotu kola extract or powder, cinnamon extract or powder, lemon extract or powder, lemon peel extract or powder, or orange extract or powder. In certain embodiments, any of these one or more flavoring agents can be present in an amount of 0.05 to 5% w/w extract or 0.1 to 0.5% w/w powder.

In certain embodiments, a toothpaste composition of the present technology further comprises 0.1 to 1% w/w of the marbling agent acacia arabica (gum Arabic).

In certain embodiments, any composition herein can be used as an additive blend, for example, in amounts of 5 to 10% w/w to improve the antimicrobial efficiency of any oral care composition (including any known commercial oral care composition, including but not limited to a toothpaste or mouthwash); in various embodiments, this can improve the antibacterial efficacy of such commercial product, for example, by causing it to exhibit 3 or more logio reduction from control bacterial counts within 30 seconds of contact.

In certain embodiments, a toothpaste composition of the present technology further comprises one or more of the following: nutmeg oil or extract; mace oil or extract; camphor powder or oil; curcumin oil or extract; or stevia extract.

In certain embodiments, the present technology is directed to an alcohol free oral spray comprising: 0.01 to 0.1% w/w thymol; 0.01 to 0.1% menthol; 0.05 to 0.3% w/w pomegranate extract; 0.05 to 0.3% grapefruit seed extract; 1 to 5% peppermint oil or peppermint extract; 1 to 5% w/w spearmint oil or extract; 2 to 20% licorice extract; 0.5 to 10% clove oil or extract; 0.5 to 5% w/w cardamom oil or extract, 5 to 15% w/w glycerin, 1 to 10% xylitol; 0.1 to 0.5% w/w ginger extract and water.

In certain embodiments, the present technology is directed to an alcohol free oral spray comprising: 0.01 to 0.1% w/w thymol; 0.01 to 0.1% menthol; 0.05 to 0.3% w/w pomegranate extract; 0.05 to 0.3% grapefruit seed extract; 0.5 to 5% peppermint oil or peppermint extract; 0.3 to 5% w/w spearmint oil or extract; 2 to 20% licorice extract; 0.5 to 10% clove oil or extract; 0.1 to 5% w/w cardamom oil or extract, 5 to 15% w/w glycerin, 1 to 15% xylitol; 0.1 to 0.5% w/w ginger extract and water.

In certain embodiments, the oral spray composition of the technology further comprises one or more of the following: nutmeg oil or extract; mace oil or extract; camphor powder or oil; curcumin oil or extract; and curcumin extract or stevia extract.

In certain embodiments, a composition herein comprises: 0.04 to 0.1% w/w thymol; 0.05 to 0.1% w/w menthol; 0.5 to 1% w/w grapefruit seed extract; 0.1 to 0.5% w/w pomegranate extract; 0.03 to 0.06% w/w peppermint oil; 0.03 to 0.08 spearmint oil; or more anti-inflammatory agents, which can be 0.01 to 0.1% w/w oil of wintergreen (methyl salicylate), 0.1 to 1% w/w calendula extract, 0.01 to 0.1% w/w zinc salicylate, 0.1 to 1% w/w aloe, or a combination thereof; 1 to 15% w/w glycerin as a solubilizer; 1 to 5% w/w natural 1,3 propanediol (available as Zemea®); 0.1 to 1% w/w caprylyl capryl glucoside (available as Plantasol®); 0.2 to 1% of one or more organic surfactants selected from: sorbitan mono laurate (available as Tween 20); sorbitan mono oleate (available as Tween 80); sorbitan oleate decylglucoside; and sorbitan sugar (poly suga mulse); wherein the ratio of caprylyl glucoside to surfactant ranges from 1:1 to 1:20 and the combined concentration of solubilizer and surfactant does not exceed 2% of the composition.

In certain embodiments, the technology herein is directed to methods of treating a wound, the methods comprising contacting the wound with any of the compositions discussed herein. In certain embodiments, the wound is an oral wound.

In certain embodiments, the technology herein is directed to a medical device coated with one or more of the compositions discussed herein.

### DETAILED DESCRIPTION

All percentages expressed herein are by weight, unless otherwise indicated.

As used herein, “low alcohol” means containing between 1 and 21% alcohol.

As used herein, “alcohol-free” means containing less than 1% alcohol.

In certain embodiments, the compositions herein are low alcohol or alcohol-free. In certain embodiments, the alcohol-free compositions contain less than 0.5% alcohol, or less than 0.25% alcohol, or less than 0.1% alcohol.

As used herein, “stable” refers to a formulation that remains intact without visibly detectable phase separation or precipitation for at least 1 year. Referring to the embodiments herein, stability can be verified by an accelerated aging test, as follows:

Temperature Variations: High temperature testing is commonly used as a predictor of long-term stability. High temperature testing at 37° C. (98° F.) and 45° C. (113° F.) are conducted. If a product is stored at 45° C. for three months (and exhibits acceptable stability), then it can be predictably stable at room temperature for two years. At the same time, the product is stored at 25° C. (77° F.) for a period of one year. A control temperature is 4° C. (39° F.) where most products will exhibit excellent stability.

Light Exposure Testing: Formulations are placed in a transparent glass bottles, next to the window for a period of 6 months. A glass jar completely covered with aluminum foil and placed at the same location serves as the control. Discoloration is studied and documented.

pH change: The pH stability of the oral rinses was documented bi-monthly for 3 months.

As used herein, “botanical” means containing active ingredients that are derived from plants, herbs or fruits or other natural sources. Throughout the disclosure, “botanical” can be an adjective, or alternatively can be a noun (for example, certain embodiments herein refer to “one or more botanicals” which refers to a botanical ingredient, such as a botanical extract or essential oil).

As used herein, “botanical extract” means a composition from a plant source (a botanical) that is prepared by soaking the botanical in a solvent (e.g., water or alcohol). A botanical extract refers to the resultant liquid, which contains the essential oil with the solvent. As described in Examples and data herein, the terminology “(100%/oil)” denotes 100% extract or oil.

In certain embodiments, the present technology is directed to a soluble (clear) and stable, alcohol-free, completely botanical oral care composition.

In various embodiments, the composition is in the form of a liquid, such as an oral rinse or mouthwash.

In certain embodiments, a composition herein contains a synergistic combination of one or more of any of the following: antimicrobial botanicals, anti-inflammatory botanicals, organic or natural solvents. In certain embodiments, a composition herein includes a combination of botanic solubilizer caprylyl capryl glucoside (for example, those known under the tradename Plantasol®) and organic surfactant sorbitan mono laurate (for example, those known under the tradename Tween 20) and in a combined amount of less than 2% w/w of the composition; or a ratio of solubilizer to surfactant from 1.1 to 1:20. One or both of these features can make the solution clear and stable, and can lead to significantly improved efficacy even over a composition containing higher concentration (more than 1%) of either the sorbitan mono laurate or the botanic solubilizer caprylyl capryl glucoside alone.

Synergistic combinations of botanicals used in the present formulations can allow the use of low concentration of botanicals, thereby minimizing or completely avoiding adverse effects from the use of higher concentrations of these ingredients. Furthermore, the addition of one or more anti-inflammatory, anti-irritant botanicals in the compositions can reduce the irritation potential of the compositions.

In certain embodiments, the compositions herein can include one or more of the following ingredients: thymol (for example, 0.01 to 1% w/w or 0.05 to 0.1% w/w), menthol (for example, 0.01 to 1% 0.05 to 0.1% w/w), grapefruit seed extract (for example, 0.5 to 1% w/w or 0.3 to 1%), pomegranate extract or oil (for example, 0.05 to 1% or 0.1 to 0.5% w/w), peppermint oil (for example, 0.01 to 0.1% w/w or 0.03 to 0.06% w/w), spearmint oil (for example, 0.01 to 0.1% w/w or 0.03 to 0.08% w/w), or one or more anti-inflammatory agents comprising oil of wintergreen (for example, methyl salicylate 0.01 to 0.1% w/w), or calendula extract (for example, 0.1 to 1% w/w) or zinc salicylate (for example, 0.01 to 0.1% w/w) or aloe (for example, 0.1 to 1%) or a combination thereof

In certain embodiments, the compositions can include one or more of the following ingredients: solubilizer glycerin (for example, 1 to 15% w/w or 5 to 13% w/w), natural 1,3 propanediol (for example, Zemea® 2 to 5% w/w), caprylyl capryl glucoside (for example, Plantasol®, in amounts of 0.2 to 1% w/w) or organic surfactant sorbitan mono laurate (for example, Tween 20 in amounts of 0.2 to 1% w/w), sorbitan mono oleate (for example, Tween 80 in amounts of 0.2 to 1% w/w), sorbitan oleate decylglucoside, sorbitan sugar (for example, Poly Suga Mulse) and combinations thereof In certain embodiments, the ratio of caprylyl capryl glucoside to surfactant ranges from 1:1 to 1:20 and the total concentration of caprylyl capryl glucoside and surfactant does not exceed 2%.

In certain embodiments, the composition further comprises 0.05 to 0.5% w/w benzoic acid, or 0.05 to 0.5% w/w sodium benzoate, or a combination thereof.

In certain embodiments, the composition further comprises one or more additional botanical ingredients, for example, clove oil, cinnamon oil, lemon grass oil, orange oil, vanilla oil, fennel oil, eucalyptol, eucalyptus oil or any combination thereof

In certain embodiments, the composition further comprises one or more sugar alcohols, for example, xylitol, sorbitol or a combination thereof (for example, 2 to 8% each, or 2.5 to 7 each, or 3 each or 4 each).

In certain embodiments, a composition herein further comprises chlorhexidine gluconate, for example, in amounts of 0.025 to 1%, or 0.05 to 0.5%.

In certain embodiments, a composition herein further comprises an artificial sweetener, for example, sucralose, either alone or in combination with dextrose or maltodextrin (for example, those available under the tradename Splenda®).

In various embodiments, the compositions herein can be in the form of a solid or liquid, for example, one suitable for oral care, including but not limited to a rinse, a spray, or a gel, a wash solution, a lotion, a cream, a sanitizer or a paste. In various embodiments, the compositions herein can be incorporated into a consumer product, including but not limited to, an oral rinse, a toothpaste, an oral patch, dental floss, a lozenge, an oral care wipe or an oral care gel.

In certain embodiments, the compositions herein can contain one or more of the following flavoring agents: clove extract or powder, cardamom extract or powder, peppermint extract, spearmint extract, licorice root extract or powder, white oak bark extract or powder, gotu kola extract or powder, cinnamon extract or powder, lemon extract or powder, lemon peel extract or powder, or orange extract or powder. In various embodiments, these can be present in amounts of 0.05 to 5% w/w or 0.1 to 0.5% w/w (in certain non-limiting examples, 0.05 to 5% w/w extract or 0.1 to 0.5% w/w powder).

In certain embodiments, the compositions herein further contain one or more of the following: one or more of the following: nutmeg oil or extract; mace oil or extract; camphor powder or oil; curcumin oil or extract; curcumin extract or stevia extract. In various embodiments, any of these can be present in amounts of, for example, 0.01 to 0.2% w/w or 0.5 to 2% w/w.

### EXAMPLE 1A

### Oral Rinse Base

An oral rinse base (“Base”) was prepared as follows:

| Ingredient | % w/w |
|---|---|
| Water | 69.4 |
| Pluronic F-127 | 1 |
| Citric acid | 0.1 |
| Sorbitol | 7 |
| Xylitol | 5 |
| Glycerin | 8 |
| Propylene glycol | 2 |
| Plantasol ® caprylyl capryl glucoside | 0.5 |
| Total Oral Rinse Base | 93 |

Oral rinses were prepared as follows (for combination with the oral rinse base):

### Oral Rinse 1

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |

Make the volume to 100gm with water.

Note: All the formulations below are adjusted to 100% with water.

Oral rinse 1 C (Oral rinse 1+0.05% chlorhexidine)

| | |
|---|---|
| Base | 93 |
| Zemea ® (1,3 propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extract | 1 |
| Chlorhexidine gluconate | 0.05 |

Oral rinse 92 (Oral rinse 1 and 0.1% pomegranate)

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |
| Pomegranate extract | 0.1 |

Oral rinse 92C (Oral rinse 92+0.05% Chlorhexidine)

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |
| Pomegranate extract | 0.1 |
| Chlorhexidine gluconate | 0.05 |

Oral rinse 93 (Oral rinse 1 and 0.2% pomegranate)

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |
| Pomegranate extract | 0.2 |

Make the volume to 100 μm with water.

Oral rinse 93-C (93+0.05% chlorhexidine)

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |
| Pomegranate extract | 0.2 |
| Chlorhexidine gluconate | 0.05 |

Oral rinse 93-C 1 (93+0.12% chlorhexidine)

| | |
|---|---|
| Base | 93 |
| Zemea ® (1,3 Propanediol) | 4 |
| Thymol | 0.07 |
| Menthol | 0.07 |
| Plantasol ® caprylyl capryl glucoside | 0.5 |
| Spearmint oil | 0.05 |
| Grapefruit Extract | 0.8 |
| Calendula extracts | 1 |
| Pomegranate extract | 0.2 |
| Chlorhexidine gluconate | 0.12 |

### Oral Rinse C

| | |
|---|---|
| Base | 93 |
| Chlorhexidine gluconate | 0.05 |
| Plantasol ® caprylyl capryl glucoside | 0.5 |

Oral Rinse C-1

| | |
|---|---|
| Base | 93 |
| Chlorhexidine gluconate | 0.12 |
| Plantasol ® caprylyl capryl glucoside | 0.5 |

Oral Rinse P

| | |
|---|---|
| Base | 93 |
| Pomegranate extract | 0.2 |
| Plantasol ® caprylyl capryl glucoside | 0.5 |

Antimicrobial efficacy of the comparative examples was tested by Rapid of kill Method 15 second exposure to bacteria.

Results:

| | Log10 reduction from control growth | | |
|---|---|---|---|
| Group | C. albicans | S. aureus | S. mutans |
| 1 | 1.25 | 2.5 | 2.8 |
| 1C | 2 | 2.8 | ND |
| 92 | 2.6 | 3 | ND |
| 92C | 3.9 | 4.2 | ND |
| 93 | 3 | 3.5 | 3.2 |
| 93C | 4.3 | 5.6 | 5 |
| 93-C1 | 4.5 | 5.8 | 5.3 |
| C | 0.4 | 0.3 | 0.5 |
| C1 | 0.5 | 0.35 | 0.6 |
| P | 0.3 | 0.3 | 0.5 |

N.D: Not determined.

Conclusion: Pomegranate extract enhances the efficacy of oral rinse containing thymol, menthol and grapefruit seed extract. Chlorohexidine synergistically enhances the efficacy of oral rinse containing pomegranate extract.

When the efficacy of Oral rinse 93 containing botanical actives and preservative level of CHX (0.05%) was compared with commercial product—higher concentration of chlorhexidine (0.12%) containing oral rinse GUM, it was equally effective as GUM against VAP pathogens. It can thus be used as an alternative to GUM without any adverse effects.

Several oral care products containing antiseptics, botanicals with and without alcohol have been marketed for oral hygiene. BOR containing thymol, menthol, pomegranate extract and grapefruit extract and chlorhexidine (92 C-93 C) showed better antibacterial efficacy when compared with the widely used other oral rinses.

The above 92 and 93 Oral rinses, while containing botanical actives and some botanical solvents, could not be made soluble (clear) and stable without the addition of synthetic surfactant, for example, poloxamers such as those available under the tradename “Pluronic®” (for example, Pluronic®-F127 or Pluronic® F-97) and solvent such as propylene glycol.

In certain embodiments, the technology disclosed herein is directed to a medical device coated with one or more of the compositions discussed herein. In certain embodiments, any of the compositions herein can be applied to an oral care device such as, for example, a toothbrush, a toothpick, a swab, an implant or dental floss.

### EXAMPLE 1B

### Effect of Concentration of Solubilizers and Surfactants Used in Oral Rinse on the Antibacterial Efficacy

In accordance with certain embodiments of the present technology, the following formulations were made and tested using the Rapid kill test method.

Rapid kill (20 seconds) test tube method described in ASTM-E2783-1198751-1 (suspension test).

In vitro rapid-kill test was carried out according to ASTM E2783-11; 10⁸ CFU mL⁻¹ microbial culture (S. aureus) were prepared in a TSA media in order to determine the antimicrobial activity of oral rinses. 0.9 mL oral rinse was kept in contact with 0.1 mL microbial cultures for 20 seconds and subsequently neutralized using drug inactivating solution (DE).

After serial dilution with DE, 0.5 mL dilutions were plated on Trypticase agar plates, incubated at 37° C. for 24 to 48 hours. Colony counts were determined and the log reduction of counts from control sample was calculated.

PBS was used as a control. In this study, an oral care product is considered effective, if the logio reduction exhibits 3.0 or higher.

Results are shown in Table 1.

TABLE 1

| | | BOR- | BOR- | BOR- | BOR- |
|---|---|---|---|---|---|
| | BOR- | PC- | PC- | PC- | PC- |
| Ingredient | PC | 7A | 7B | 7C | 7D |
| Water | 69.79 | 73.72 | 73.72 | 74.02 | 74.02 |
| Pomegranate extract | 0.1 | 0.2 | 0.2 | 0.2 | 0.2 |
| Sorbitan monolaurate | 0 | 1.5 | 0 | 0.7 | 0.4 |
| botanic solubilizer | 0.5 | 0 | 1.5 | 0.7 | 0.6 |
| caprylyl capryl | | | | | |
| glucoside | | | | | |
| poloxamer (Pluronic ® | 1 | 0 | 0 | 0 | 0 |
| F-127) | | | | | |
| Propylene Glycol | 2 | 0 | 0 | 0 | 0 |
| Benzoic Acid | 0 | 0.1 | 0.1 | 0.1 | 0.1 |
| Sodium benzoate | 0 | 0.1 | 0.1 | 0.1 | 0.1 |
| Sorbitol | 7 | 4 | 4 | 4 | 4 |
| Xylitol | 5 | 3 | 3 | 3 | 3 |
| Grapefruit Seed | 0.8 | 0.8 | 0.8 | 0.8 | 0.8 |
| Extract | | | | | |
| propanediol | 4 | 4 | 4 | 4 | 4 |
| Glycerin | 8 | 12.5 | 12.5 | 12.5 | 12.5 |
| Thymol | 0.1 | 0.08 | 0.08 | 0.08 | 0.08 |
| Menthol | 0.06 | 0.07 | 0.07 | 0.07 | 0.07 |
| Peppermint oil | 0 | 0.04 | 0.04 | 0.04 | 0.04 |
| Methyl Salicylate | 0 | 0.05 | 0.05 | 0.05 | 0.05 |
| Spearmint | 0.05 | 0.04 | 0.04 | 0.04 | 0.04 |
| Calendula Extracts | 1 | 0 | 0 | 0 | 0 |
| Citric Acid | 0.1 | 0 | 0 | 0 | 0 |
| Sucralose + | 0.5 | 0 | 0 | 0 | 0 |
| dextrose and | | | | | |
| maltodextrin | | | | | |
| Total | 100 | 100 | 100 | 100 | 100 |
| Clarity of | Clear | Clear | Clear | Clear | Clear |
| Formulation | | | | | |
| Log₁₀ Reduction | 3.5 | 2.6 | 2.8 | 4.0 | 4.0 |

Conclusion: Composition containing more than 1.0% sorbitan mono laurate or botanic solubilizer caprylyl capryl glucoside reduces the antibacterial efficacy. However, addition of combination of sorbitan mono laurate and botanic solubilizer caprylyl capryl glucoside at 1 to 1.4% concentration level and ratio of 1.1 to 1:1.5 makes the solution clear and the efficacy is more than the other groups.

### EXAMPLE 2

Another formulation was prepared, which contained lower concentrations of Thymol and Menthol and eucalyptus oil and eucalyptol (BOR-PC-8). BOR-PC-7D and BOR-PC-8 were evaluated for their antimicrobial efficacy and compared with that of other commercial oral rinses. Results are shown in Table 2.

TABLE 2

| Ingredient | BOR-PC-8 |
|---|---|
| Water | 73.87 |
| Pomegranate extract | 0.2 |
| Sorbitan monolaurate | 0.5 |
| solubilizer caprylyl capryl glucoside | 0.6 |
| Benzoic Acid | 0.1 |
| Sodium benzoate | 0.1 |
| Sorbitol | 4 |
| Xylitol | 3 |
| Grapefruit Seed Extract | 0.8 |
| Propanediol | 4 |
| Glycerin | 12.5 |
| Thymol | 0.064 |
| Menthol | 0.042 |
| Peppermint oil | 0.04 |
| Methyl Salicylate | 0.06 |
| Spearmint | 0.04 |
| Eucalyptus Oil | 0.08 |
| Total | 100 |

### EXAMPLE 3

### Comparison of Ingredients in BOR and other Commercial Formulation which has Botanical and Synthetic Ingredients as well as those that have only Botanical Ingredients

TABLE 3A

| | Composition | | | | | | |
|---|---|---|---|---|---|---|---|
| | Listerine Zero | % | Therabreath | BOR 92 | % | BOR 92-PC7 | % |
| Synthetic | None | | Unknown | None | | None | |
| Antimicrobials | | | | | | | |
| Botanicals | Thymol | 0.02 | Unknown | Thymol | 0.10 | Thymol | 0.08 |
| | Peppermint oil | 0.08 | | Pomegranate extract | 0.10 | Pomegranate extract | 0.20 |
| | Anise oil | 0.014 | | Grape Fruit Seed Extract | 0.80 | Grape Fruit Seed Extract | 0.80 |
| | Eugenol | 0.006 | | Calendula Extracts | 1.00 | Peppermint oil | 0.04 |
| | Menthol | 0.06 | | Spearmint | 0.05 | Spearmint | 0.04 |
| | Methyl Salicylate | 0.11 | | Menthol | 0.06 | Menthol | 0.07 |
| | | | | | | Methyl Salicylate | 0.05 |
| Synthetic | Pluronic 105 | 0.5 | PEG-40 castor oil | Pluronic F-127 | 1.00 | None | |
| Solvents/ | Benzyl alcohol | 0.04 | | | | | |
| Solubelizers/ | Polyethylene Glycol | 0.5 | | | | | |
| Surfactants | Sodium Lauryl Sulphate | 0.21 | | | | | |
| Natural | Glycerine | 7.5 | Unknown | Zemea | 4.00 | Sorbitan monolaurate | 0.40 |
| Solvents/ | | | | Propylene Glycol | 2.00 | Zemea | 4.00 |
| Solubelizers/ | | | | Caprylyl capril glucoside | 0.50 | Glycerin | 12.50 |
| Surfactants | | | | Glycerine | 8.00 | Caprylyl capril glucoside | 0.60 |
| Others | Benzoic Acid | 0.025 | Citric Acid | Citric Acid | 0.10 | Benzoic Acid | 0.10 |
| | Sodium Saccharin | 0.05 | Sodium Benzoate | Sorbitol | 7.00 | Sodium Benzoate | 0.10 |
| | Zinc Chloride | 0.005 | Sodium Bicarbonate | Xylitol | 5.00 | Sorbitol | 4.00 |
| | Sodium Benzoate | 0.18 | Sodium chlorite | Splenda | 0.50 | Xylitol | 3.00 |
| | Water | 90.7 | Tetrasodium EDTA | Water | 69.79 | Water | 74.02 |
| | | | Water | | | | |

Completely botanical mouthwash formulations and ranges

TABLE 3B

| Mouthwash 92-PC | Percentage |
|---|---|
| Water | 73.845 |
| Pomegranate extract | 0.2 |
| sorbitan monolaurate | 0.5 |
| Benzoic Acid | 0.1 |
| Sorbitol | 4 |
| Xylitol | 3 |
| Grapefruit Seed Extract | 0.5 |
| Calendula Extracts | 1 |
| 1,3 propanediol | 4 |
| glycerin | 12 |
| Thymol | 0.07 |
| Menthol | 0.07 |
| Caprylyl capryl glucoside | 0.6 |
| Peppermint oil | 0.03 |
| Methyl Salicylate | 0.015 |
| Clove Oil | 0.01 |
| Spearmint | 0.05 |
| Color | 0.01 |
| Total | 100 |

### EXAMPLE 4

### Ingredient Ranges of Completely Botanical Oral Rinses BOR-PC-92, BOR-PC-7D and BOR PC-8

BOR-PC-92

TABLE 4A

| Mouthwash 92-PC | Percentage |
|---|---|
| Water | 60-75 |
| Pomegranate extract | 0.1-1 |
| sorbitan monolaurate (Tween 20) | 0.2-0.8 |
| Benzoic Acid | 0.1-0.2 |
| Sodium Benzoate | 0.1-0.2 |
| Sorbitol | 2-8 |
| Xylitol | 2-8 |
| Grapefruit Seed Extract | 0.5-2 |
| Calendula Extract | 0.01-1 |
| Propanediol | 2-5 |
| glycerin | 10-15 |
| Thymol | 0.05-0.2 |
| Menthol | 0.05-0.2 |
| Caprylyl capryl | 0.2-0.8 |
| Peppermint oil | 0.03-0.1 |
| Methyl Salicylate | 0.01-0.1 |
| Clove Oil | 0-0.03 |
| Spearmint | 0.03-0.1 |
| Total | 100 |

TABLE 4B

| Mouthwash 92-PC-7 | Range Percentage |
|---|---|
| Water | 65-75 |
| Pomegranate extract/oil | 0.1-0.5 |
| sorbitan monolaurate | 0.3-0.8 |
| Benzoic Acid | 0.1-0.2 |
| Sodium benzoate | 0.1-0.2 |
| Sorbitol | 4-8 |
| Xylitol | 2-8 |
| Grapefruit Seed Extract | 0.3-1 |
| Propanediol | 2-5 |
| Glycerin | 5-15 |
| solubilizer caprylyl capryl glucoside | 0.3-0.8 |
| Thymol | 0.05-0.08 |
| Menthol | 0.05-0.08 |
| Peppermint oil | 0.03-0.06 |
| Methyl Salicylate | 0.01-0.1 |
| Spearmint oil | 0.03-0.06 |
| Total | 100 |

TABLE 4C

| Mouthwash 92-PC-7 | Percentage |
|---|---|
| Water | 74.02 |
| Pomegranate extract | 0.2 |
| sorbitan monolaurate | 0.4 |
| Benzoic Acid | 0.1 |
| Sodium benzoate | 0.1 |
| Sorbitol | 4 |
| Xylitol | 3 |
| Grapefruit Seed Extract | 0.8 |
| Propanediol | 4 |
| Glycerin | 12.5 |
| solubilizer caprylyl capryl glucoside | 0.6 |
| Thymol | 0.08 |
| Menthol | 0.07 |
| Peppermint oil | 0.04 |
| Methyl Salicylate | 0.05 |
| Spearmint | 0.04 |
| Total | 100 |

TABLE 4D

| Mouthwash (92-PC-8) | Range Percentage |
|---|---|
| Water | 65-75 |
| Pomegranate extract | 0.1-0.5 |
| Sorbitan monolaurate | 0.3-0.8 |
| Benzoic Acid | 0.1-0.2 |
| Sodium benzoate | 0.1-0.2 |
| Sorbitol | 4-8 |
| Xylitol | 2-8 |
| Grapefruit Seed Extract | 0.3-1 |
| Propanediol | 2-5 |
| Glycerin | 5-15 |
| solubilizer caprylyl capryl glucoside | 0.3-0.8 |
| Thymol | 0.05-0.08 |
| Menthol | 0.05-0.08 |
| Peppermint oil | 0.03-0.06 |
| Methyl Salicylate | 0.01-0.1 |
| Spearmint | 0.03-0.06 |
| Eucalyptus Oil/Eucalyptol | 0.05-0.1 |
| Total | 100 |

TABLE 4E

| Mouthwash (92-PC-8) | Percentage |
|---|---|
| Water | 73.881 |
| Pomegranate extract | 0.2 |
| Sorbitan monolaurate | 0.5 |
| Benzoic Acid | 0.1 |
| Sodium benzoate | 0.1 |
| Sorbitol | 4 |
| Xylitol | 3 |
| Grapefruit Seed Extract | 0.8 |
| Propanediol | 4 |
| Glycerin | 12.5 |
| solubilizer caprylyl capryl glucoside | 0.6 |
| Thymol | 0.064 |
| Menthol | 0.042 |
| Peppermint oil | 0.04 |
| Methyl Salicylate | 0.06 |
| Spearmint | 0.04 |
| Eucalyptus Oil | 0.083 |
| Total | 100 |

TABLE 4F

| Mouthwash (92-PC-9) | Percentage |
|---|---|
| Water | 73.861 |
| Pomegranate extract | 0.2 |
| Sorbitan monolaurate | 0.5 |
| Benzoic Acid | 0.1 |
| Sodium benzoate | 0.1 |
| Sorbitol | 4 |
| Xylitol | 3 |
| Grapefruit Seed Extract | 0.8 |
| Propanediol | 4 |
| Glycerin | 12.5 |
| solubilizer caprylyl capryl glucoside | 0.6 |
| Thymol | 0.064 |
| Menthol | 0.042 |
| Peppermint oil | 0.04 |
| Methyl Salicylate | 0.06 |
| Spearmint | 0.04 |
| Eucalyptus Oil | 0.083 |
| Zinc salicylate | 0.02 |
| Total | 100 |

TABLE 4G

| Mouthwash (92-PC-8 A) | Range Percentage |
|---|---|
| Water | 65-75 |
| Pomegranate extract | 0.1-0.5 |
| Sorbitan monolaurate | 0.3-0.8 |
| Benzoic Acid | 0.1-0.2 |
| Sodium benzoate | 0.1-0.2 |
| Sorbitol | 4-8 |
| Xylitol | 2-8 |
| Grapefruit Seed Extract | 0.3-1 |
| 1,3 propanediol | 2-5 |
| Glycerin | 5-15 |
| Plantasol ® caprylyl capryl glucoside | 0.3-0.8 |
| Thymol | 0.05-0.08 |
| Menthol | 0.05-0.08 |
| Peppermint oil | 0.03-0.06 |
| Methyl Salicylate | 0.01-0.1 |
| Spearmint | 0.03-0.06 |
| Eucalyptol | 0.05-0.1 |
| Total | 100 |

TABLE 4H

| Mouthwash (Batch 92-PC-8B) | Percentage |
|---|---|
| Water | 72.862 |
| Pomegranate extract | 0.2 |
| Sorbitan monolaurate | 0.5 |
| Benzoic Acid | 0.1 |
| Sodium benzoate | 0.1 |
| Sorbitol | 4 |
| Xylitol | 4 |
| Grapefruit Seed Extract | 0.8 |
| 1,3 propanediol | 4 |
| Glycerin | 12.5 |
| Plantasol ® caprylyl capryl glucoside | 0.6 |
| Thymol | 0.064 |
| Menthol | 0.042 |
| Peppermint oil | 0.04 |
| Methyl Salicylate | 0.06 |
| Spearmint | 0.04 |
| Eucalyptol | 0.092 |
| Total | 100 |

TABLE 4I

| Mouthwash 92-PC-7B | Percentage |
|---|---|
| Water | 72.075 |
| Pomegranate extract | 0.2 |
| sorbitan monolaurate | 0.6 |
| Benzoic Acid | 0.1 |
| Sodium benzoate | 0.1 |
| Sorbitol (70% solution) | 5.72 |
| Xylitol | 3 |
| Grapefruit Seed Extract | 0.8 |
| 1,3-Propanediol | 4 |
| Glycerin | 12.5 |
| Caprylyl capryl glucoside | 0.6 |
| Thymol | 0.08 |
| Menthol | 0.07 |
| Peppermint oil | 0.04 |
| Methyl Salicylate | 0.05 |
| Spearmint | 0.04 |
| Natural color | 0.025 |
| Total | 100 |

In various embodiments, these formulations can contain natural coloring agents, including copper salts, or chlorophyllin botanical extracts or combination thereof.

### EXAMPLE 5

### Antimicrobial Efficacy of BOR Formulations vs. Commercially Available Mouth Washes using Plate Method

Plate method: After 1-minute exposure of 0.5 mL of oral rinse to colonized bacteria on agar plate, the reduction in bacterial colonies was determined in the rinse solution.

Detailed Method: 0.3 mL of 10⁷ CFU mL⁻¹ microbial cultures were seeded on TSA plates and incubated for 4 h at 37° C. for microbes to colonize. Then, 0.5 mL of oral rinse or PBS (control) was added and spread on the surface of the plate during 10 s of plating.

After 1 minute, 4.5 mL of DE (drug neutralizer) was added to inactivate the active ingredients, swirled to cover the whole plate with a glass spreader and collected in a culture tube for 10 s. An additional 4.5 mL of DE was added on the agar plate and the process was repeated.

Finally, the fluid was transferred into the same culture tube and mixed well and left for at least 5 min. After serial dilution with DE, 0.5 mL of aliquot was spread on TSA plate (brain heart infusion agar plate was used for S. mutans) and incubated at 37° C. for 18 h. The colony counts on the plates were determined.

Results:

Log₁₀ Reduction of BOR compositions vs. commercially available mouth wash using the plate method. The results were taken after 1 minute.

TABLE 5

| | Plate Method - colonized | | | | |
|---|---|---|---|---|---|
| | | | | BOR- | BOR- |
| | Listerine ® | | BOR- | PC- | PC- |
| Bacteria | Zero | TheraBreath | 92 | 7D | 8 |
| S. mutans | 4.00 | 0.10 | 4.00 | 4.00 | 4.20 |
| C. albicans | 3.21 | 0.10 | 2.85 | 4.97 | 5.00 |
| S. aureus | 3.53 | 0.50 | 3.00 | 4.00 | 4.60 |
| P. aeruginosa | 0.10 | 0.00 | 4.08 | 3.50 | 3.80 |
| A. | N.D. | 0.50 | N.D. | N.D. | 5.80 |
| actinomycetmcomitans | | | | | |

### EXAMPLE 6

In various embodiments, the compositions herein can be used to prepare completely natural toothpaste and gels. Several toothpaste formulations were prepared by directly incorporating the composition into toothpaste base and testing using zone of inhibition.

The following Natural Toothpastes A and B were prepared and tested.

Natural toothpaste A-1 (Ranges)

| Ingredient | Range | Type |
|---|---|---|
| Water | 5-20 | |
| Xylitol | 4-20 | |
| Hydrated Silica | 0-20 | |
| Xanthan Gum | 0.1-2 | |
| Glycerin | 8-30 | |
| Sorbitol | 4-20 | |
| Cocoamidopropyl Betaine | 0.8-5 | |
| Calcium Carbonate | 0.5-20 | |
| Sodium Bicarbonate | 0.1-40 | |
| Organic Stevia | 0.1-1 | |
| Sea Salt | 0.1-5 | Natural |
| Coconut Oil | 0.5-20 | Botanical |
| 1,3 propanediol | 2-20 | Botanical |
| Grapefruit Seed Extract | 0.5-2 | Botanical |
| Peppermint oil | 0.03-1 | Botanical |
| Spearmint oil | 0.03-1 | Botanical |
| Benzoic acid | 0.1-0.5 | Preservative |
| Sodium Benzoate | 0.1-0.5 | Preservative |
| Zinc Salicylate | 0-0.5 | Anti-inflammatory |
| Pomegranate extract | 0.1-1 | Botanical |
| Clove bud oil | 0.05-1 | Botanical |
| Eucalyptol | 0.05-1 | Botanical |
| Methyl salicylate | 0.01-1 | Anti-inflammatory |
| Sorbitan monolaurate | 0.2-1 | Botanical |
| Caprylyl capryl glucoside | 0.2-1 | Botanical |
| Thymol | 0.05-1 | |
| Menthol | 0.05-1 | |
| Benzyl alcohol | 0.01-2 | |
| Carrageenan (1.6% in water) | 1-25 | |
| Total | | |
| Flavoring Agents | | |
| Clove extract/clove oil | 0.1-0.5 | |
| Fennel extract/fennel oil | 0.1-0.5 | |
| Peppermint extract | 1-5 | |
| Orange peel powder | 0.1-1 | |
| Licorice extract | 2-10 | |
| Lemon peel powder | 0.5-2 | |
| Spearmint extract | 1-5 | |
| Anti-irritant/Anti- | | |
| inflammatory agents | | |
| Calendula extract | 0.5-2 | |
| Zinc salicylate | 0.1-0.5 | |
| Aloe gel extract | 0.5-2 | |

Natural toothpaste A-2 (Ranges)

| | Range | |
|---|---|---|
| Ingredient | % w/w | Type |
| Water | 5-35 | |
| Xylitol | 4-25 | |
| Hydrated Silica | 0-20 | |
| Xanthan Gum | 0.1-2 | |
| Glycerin | 8-50 | |
| Sorbitol | 4-20 | |
| Cocoamidopropyl Betaine | 0.01-5 | |
| Calcium Carbonate | 0.1-45 | |
| Sodium Bicarbonate | 0.1-40 | |
| Organic Stevia | 0.1-3 | |
| Sea Salt | 0.1-5 | Botanical |
| Coconut Oil | 0.5-20 | Botanical |
| 1,3 propanediol | 2-20 | Botanical |
| Grapefruit Seed Extract | 0.5-2 | Botanical |
| Peppermint oil | 0.03-1 | Botanical |
| Spearmint oil | 0.03-1 | Botanical |
| Benzoic acid | 0.1-0.5 | Preservative |
| Sodium Benzoate | 0.1-0.5 | Preservative |
| Zinc Salicylate | 0-0.5 | Anti-inflammatory |
| Pomegranate extract | 0.1-1 | Botanical |
| Clove bud oil | 0.05-1 | Botanical |
| Eucalyptol | 0.05-1 | Botanical |
| Methyl salicylate | 0.01-1 | Anti-inflammatory |
| Sorbitan monolaurate | 0.2-1 | Botanical |
| Caprylyl capryl glucoside | 0.2-1 | Botanical |
| Thymol | 0.05-1 | |
| Menthol | 0.05-1 | |
| Benzyl alcohol | 0.01-2 | |
| Carrageenan (1.6% in water) | 1-25 | |
| Carrageenan powder | 0.01-5 | |
| Methyl Cellulose | 0.01-1 | |
| Saponin | 0.1-5 | |
| Gum Arabic | 0.1-1 | |
| Other Agents | | |
| Clove (powder or oil) | 0.1-0.5 | |
| Fennel (powder or oil) | 0.1-0.5 | |
| Peppermint flavor | 0-5-5 | |
| Orange peel powder | 0.1-1 | |
| Gotu Kola powder | 0.1-0.5 | |
| White Oak Bark powder | 0.1-0.5 | |
| Cinnamon Powder | 0.1-0.5 | |
| Cardamom Powder | 0.1-0.5 | |
| Cardamom Ext | 0.1-0.5 | |
| Licorice (Root) Powder | 0.1-0.5 | |
| Lemon peel powder | 0.1-0.5 | |
| Peppermint leaf extract | 0.1-1 | |
| Anti-irritant/Anti- | | |
| inflammatory agents | | |
| Calendula extract | 0.5-2 | |
| Zinc salicylate | 0.1-0.5 | |
| Aloe gel extract | 0.5-1 | |

TABLE 6

| | TP-NB-4 |
|---|---|
| Ingredient | % w/w |
| Water | q.s. |
| Xylitol | 8 |
| Hydrated Silica | — |
| Xanthan Gum | 2 |
| Glycerin | 15 |
| Sorbitol | 8 |
| Cocoamidopropyl Betaine | 1 |
| Calcium Carbonate | 40 |
| Sodium Bicarbonate | — |
| Organic Stevia | — |
| Sea Salt | 1 |
| Coconut Oil | 10 |
| 1,3 propanediol | 2 |
| Grapefruit Seed Extract | 0.8 |
| Peppermint oil | 0.1 |
| Spearmint oil | 0.1 |
| Benzoic acid | 0.1 |
| Sodium Benzoate | 0.2 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | — |
| Carrageenan (1.6% in water) | — |

TABLE 7

| | TP-NB-5 |
|---|---|
| Ingredient | % w/w |
| Water | q.s. |
| Xylitol | 8 |
| Hydrated Silica | — |
| Xanthan Gum | 0.1 |
| Glycerin | 15 |
| Sorbitol | 8 |
| Cocoamidopropyl Betaine | — |
| Calcium Carbonate | 27 |
| Sodium Bicarbonate | — |
| Organic Stevia | — |
| Sea Salt | 1 |
| Coconut Oil | 5 |
| 1,3 propanediol | 12 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | 20 |

TABLE 8

| | TP-NB-6 |
|---|---|
| Ingredient | % w/w |
| Water | q.s. |
| Xylitol | 10 |
| Hydrated Silica | — |
| Xanthan Gum | 1 |
| Glycerin | 15 |
| Sorbitol | 10 |
| Cocoamidopropyl Betaine | 2 |
| Calcium Carbonate | 5 |
| Sodium Bicarbonate | 30 |
| Organic Stevia | — |
| Sea Salt | 1 |
| Coconut Oil | — |
| 1,3 propanediol | 8 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | — |

TABLE 9

| | TP-NB-7 |
|---|---|
| Ingredient | % w/w |
| Water | q.s. |
| Xylitol | 8 |
| Hydrated Silica | 15 |
| Xanthan Gum | 1.2 |
| Glycerin | 20 |
| Sorbitol | 16 |
| Cocoamidopropyl Betaine | 1.5 |
| Calcium Carbonate | 5 |
| Sodium Bicarbonate | — |
| Organic Stevia | 1 |
| Sea Salt | 1 |
| Coconut Oil | 10 |
| 1,3 propanediol | 5.9 |
| Grapefruit Seed Extract | 0.8 |
| Peppermint oil | 0.1 |
| Spearmint oil | 0.1 |
| Benzoic acid | 0.1 |
| Sodium Benzoate | 0.2 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | — |
| Carrageenan (1.6% in water) | — |

TABLE 10

| | TP-NB-8 |
|---|---|
| Ingredient | % w/w |
| Water | q.s. |
| Xylitol | 8 |
| Hydrated Silica | 15 |
| Xanthan Gum | 1.2 |
| Glycerin | 20 |
| Sorbitol | 16 |
| Cocoamidopropyl Betaine | 1.5 |
| Calcium Carbonate | 5 |
| Sodium Bicarbonate | — |
| Organic Stevia | 1 |
| Sea Salt | 1 |
| Coconut Oil | 5 |
| 1,3 propanediol | 12 |
| Grapefruit Seed Extract | 0.8 |
| Peppermint oil | 0.1 |
| Spearmint oil | 0.1 |
| Benzoic acid | 0.1 |
| Sodium Benzoate | 0.2 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | — |
| Carrageenan (1.6% in water) | — |

TABLE 11

| | TP-NB-8B |
|---|---|
| Ingredient | % w/w |
| Water | q.s. |
| Xylitol | 8 |
| Hydrated Silica | 15 |
| Xanthan Gum | 1.2 |
| Glycerin | 20 |
| Sorbitol | 16 |
| Cocoamidopropyl Betaine | 1.5 |
| Calcium Carbonate | 5 |
| Sodium Bicarbonate | — |
| Organic Stevia | 1 |
| Sea Salt | 1 |
| Coconut Oil | 5 |
| 1,3 propanediol | 12 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | — |

TABLE 12

| | TP-NB-8C |
|---|---|
| Ingredient | % w/w |
| Water | q.s. |
| Xylitol | 8 |
| Hydrated Silica | 15 |
| Xanthan Gum | 1.2 |
| Glycerin | 20 |
| Sorbitol | 16 |
| Cocoamidopropyl Betaine | 1.5 |
| Calcium Carbonate | 5 |
| Sodium Bicarbonate | — |
| Organic Stevia | 1 |
| Sea Salt | 1 |
| Coconut Oil | 5 |
| 1,3 propanediol | 12 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | — |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | — |

TABLE 13

| | TP-NB-9 |
|---|---|
| Ingredient | % w/w |
| Water | q.s. |
| Xylitol | 8 |
| Hydrated Silica | 15 |
| Xanthan Gum | 1.2 |
| Glycerin | 20 |
| Sorbitol | 16 |
| Cocoamidopropyl Betaine | 1.5 |
| Calcium Carbonate | 5 |
| Sodium Bicarbonate | — |
| Organic Stevia | 1 |
| Sea Salt | 1 |
| Coconut Oil | 5 |
| 1,3 propanediol | 12 |
| Grapefruit Seed Extract | 0.8 |
| Peppermint oil | 0.1 |
| Spearmint oil | 0.1 |
| Benzoic acid | 0.1 |
| Sodium Benzoate | 0.2 |
| Zinc Salicylate | — |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | — |

TABLE 14

| | TP-NB-10 |
|---|---|
| Ingredient | % w/w |
| Water | q.s. |
| Xylitol | 15 |
| Hydrated Silica | 20 |
| Xanthan Gum | 1 |
| Glycerin | 25 |
| Sorbitol | 5 |
| Cocoamidopropyl Betaine | 1 |
| Calcium Carbonate | 3 |
| Sodium Bicarbonate | — |
| Organic Stevia | 1 |
| Sea Salt | 1 |
| Coconut Oil | — |
| 1,3 propanediol | 9.7 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | — |

TABLE 15A

| | TP-NB-11 |
|---|---|
| Ingredient | % w/w |
| Water | q.s. |
| Xylitol | 12 |
| Hydrated Silica | 10 |
| Xanthan Gum | 1 |
| Glycerin | 20 |
| Sorbitol | 12 |
| Cocoamidopropyl Betaine | 1 |
| Calcium Carbonate | 15 |
| Sodium Bicarbonate | — |
| Organic Stevia | 1 |
| Sea Salt | 1 |
| Coconut Oil | — |
| 1,3 propanediol | 5 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan (1.6% in water) | 10 |

### EXAMPLE 7

The toothpaste formulations were tested using zone of inhibition study against S. aureus. An agar plate was inoculated with 0.3 mL of 10⁸ CFU/mL S. aureus and allowed to dry for 10 minutes. Afterwards, wells were prepared using a cork borer and 0.1mL of test toothpaste as well as control (TOM'S OF MAINE®) was introduced into the agar plate and incubated overnight. The zone of in3hibition was measured.

Selected results are shown in Table 15B.

### EXAMPLE 8

Formulation of natural toothpaste B was prepared.

TABLE 16

| | TP-NB-B1 |
|---|---|
| Ingredient | % w/w |
| Xylitol | 10 |
| Glycerin | 15 |
| Sorbitol | 10 |
| Calcium Carbonate | 1 |
| Sodium Bicarbonate | 40 |
| Sea Salt | 1 |
| Coconut Oil | 10 |
| 1,3 propanediol | 5.7 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Licorice Extract | 1 |
| Peppermint Extract | 1 |
| Spearmint Extract | 1 |
| Clove extract | 3 |

TABLE 17

| | TP-L3-P |
|---|---|
| Ingredient | % w/w |
| Water | 24 |
| Xylitol | 10 |
| Glycerin | 35.1 |
| Sorbitol | 5 |
| Calcium Carbonate | 10 |
| Sodium Bicarbonate | 0.1 |
| Organic Stevia | 2 |
| Sea Salt | 1 |
| 1,3 propanediol | 2 |
| Grapefruit Seed Extract | 0.8 |
| Peppermint oil | 0.2 |
| Spearmint oil | 0.2 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.3 |
| Benzyl alcohol | 1 |
| Carrageenan | 2 |
| Methyl Cellulose | 0.2 |
| Saponin | 2 |
| Peppermint Flavor | 1.5 |
| Clove Extract | 0.2 |
| Cardamom Powder | 0.2 |
| Total | 100 |

TABLE 18

| | TP-L3-G (Gel) |
|---|---|
| Ingredient | % w/w |
| Water | 24 |
| Xylitol | 10 |
| Glycerin | 45.1 |
| Sorbitol | 5 |
| Sodium Bicarbonate | 0.1 |
| Organic Stevia | 2 |
| Sea Salt | 1 |
| 1,3 propanediol | 2 |
| Grapefruit Seed Extract | 0.8 |
| Peppermint oil | 0.2 |
| Spearmint oil | 0.2 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.3 |
| Benzyl alcohol | 1 |
| Carrageenan | 2 |
| Methyl Cellulose | 0.2 |
| Saponin | 2 |
| Peppermint Flavor | 1.5 |
| Clove Extract | 0.2 |
| Cardamom Powder | 0.2 |
| Total | 100 |

TABLE 19

| | TP-LN-2 |
|---|---|
| Ingredient | % w/w |
| Water | 13 |
| Xylitol | 10 |
| Hydrated Silica | 10 |
| Xanthan Gum | 1 |
| Glycerin | 35 |
| Sorbitol | 5 |
| Cocoamidopropyl Betaine | 1 |
| Calcium Carbonate | 2 |
| Organic Stevia | 1 |
| Sea Salt | 1 |
| 1,3 propanediol | 11 |
| Grapefruit Seed Extract | 0.8 |
| Peppermint oil | 0.2 |
| Spearmint oil | 0.2 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.3 |
| Benzyl alcohol | 1 |
| Carrageenan | 1 |
| Methyl Cellulose | 0.2 |
| Saponin | 2 |
| Peppermint Flavor | 1.5 |
| Clove Extract | 0.2 |
| Cardamom Powder | 0.2 |
| Total | 100 |

TABLE 20

| | TP-L-4B |
|---|---|
| Ingredient | % w/w |
| Water | 25 |
| Xylitol | 20 |
| Glycerin | 20 |
| Sorbitol | 5 |
| Calcium Carbonate | 20 |
| Sea Salt | 1 |
| 1,3 propanediol | 2 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan | 1 |
| Licorice Powder | 0.2 |
| Cinnamon Powder | 0.2 |
| Clove Powder | 0.2 |
| Lemon Peel Powder | 0.2 |
| Cardamom Ext | 0.2 |
| Gum Arabic | 0.2 |
| Total | 100 |

TABLE 21

| | TP-L-5B |
|---|---|
| Ingredient | % w/w |
| Water | 25 |
| Xylitol | 20 |
| Glycerin | 17 |
| Sorbitol | 10 |
| Calcium Carbonate | 10 |
| Sodium Bicarbonate | 1 |
| Sea Salt | 2 |
| 1,3 propanediol | 7.9 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan | 1.5 |
| Licorice Powder | 0.2 |
| Cinnamon Powder | 0.2 |
| Clove Powder | 0.2 |
| Lemon Peel Powder | 0.2 |
| Cardamom Ext | 0.2 |
| Gum Arabic | 0.3 |
| Total | 100 |

TABLE 22

| | TP-L-5C |
|---|---|
| Ingredient | % w/w |
| Water | 25 |
| Xylitol | 20 |
| Hydrated Silica | 5 |
| Glycerin | 12 |
| Sorbitol | 10 |
| Calcium Carbonate | 10 |
| Sodium Bicarbonate | 1 |
| Sea Salt | 2 |
| 1,3 propanediol | 7.9 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Carrageenan | 1.5 |
| Licorice Powder | 0.2 |
| Cinnamon Powder | 0.2 |
| Clove Powder | 0.2 |
| Lemon Peel Powder | 0.2 |
| Cardamom Ext | 0.2 |
| Gum Arabic | 0.3 |
| Total | 100 |

TABLE 23

| | TP-LN-4 |
|---|---|
| Ingredient | % w/w |
| Water | 21 |
| Xylitol | 15 |
| Hydrated Silica | 15 |
| Xanthan Gum | 1 |
| Glycerin | 25 |
| Sorbitol | 5 |
| Cocoamidopropyl Betaine | 1 |
| Calcium Carbonate | 3 |
| Sodium Bicarbonate | 1 |
| Organic Stevia | 1 |
| Sea Salt | 2 |
| 1,3 propanediol | 4 |
| Grapefruit Seed Extract | 0.8 |
| Peppermint oil | 0.1 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl salicylate | 0.1 |
| Sorbitan monolaurate | 0.5 |
| Caprylyl capryl glucoside | 0.5 |
| Thymol | 0.1 |
| Menthol | 0.1 |
| Benzyl alcohol | 1 |
| Licorice Powder | 0.2 |
| Cinnamon Powder | 0.2 |
| Clove Powder | 0.2 |
| Lemon Peel Powder | 0.2 |
| Cardamom Ext | 0.2 |
| Gum Arabic | 0.3 |
| Total | 100 |

In certain embodiments, the following additional toothpaste compositions were formulated, and these fall within the scope of the present disclosure.

TABLE 24

| | TP-L-6 |
|---|---|
| Ingredient | % w/w |
| Water | 24 |
| Xylitol | 10 |
| Glycerin | 18.7 |
| Sorbitol | 5 |
| Calcium Carbonate | 30 |
| Organic Stevia | 1 |
| Sea Salt | 1 |
| Zemea ® propanediol | 2 |
| Grapefruit Seed Extract | 0.8 |
| Peppermint oil | 0.3 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl Salicylate | 0.1 |
| Sorbitan monolaurate | 0.5 |
| Capryl caprylil Glucoside | 0.5 |
| Thymol | 0.1 |
| Menthol | 0.3 |
| Benzyl alcohol | 1 |
| Carrageenan | 1 |
| Peppermint Flavor | 1.5 |
| Licorice (Root) Powder | 0.2 |
| Lemon Peel Powder | 0.2 |
| Cardamom Ext | 0.2 |
| Gum Arabic | 0.3 |
| White Oak Bark | 0.2 |
| Total | 100 |

TABLE 25

| | TP-L-6B |
|---|---|
| Ingredient | % w/w |
| Water | 24 |
| Xylitol | 10 |
| Glycerin | 19.3 |
| Sorbitol | 5 |
| Calcium Carbonate | 30 |
| Organic Stevia | 1 |
| Sea Salt | 1 |
| Zemea ® propanediol | 2 |
| Grapefruit Seed Extract | 0.8 |
| Peppermint oil | 0.3 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl Salicylate | 0.1 |
| Sorbitan monolaurate | 0.5 |
| Capryl caprylil Glucoside | 0.5 |
| Thymol | 0.1 |
| Menthol | 0.3 |
| Benzyl alcohol | 1 |
| Carrageenan | 1 |
| Peppermint Flavor | 1.5 |
| Cardamom Ext | 0.2 |
| Gum Arabic | 0.3 |
| Total | 100 |

TABLE 26

| | TP-LN-5 |
|---|---|
| Ingredient | % w/w |
| Water | 21.7 |
| Xylitol | 15 |
| Hydrated Silica | 15 |
| Xanthan Gum | 1 |
| Glycerin | 25 |
| Sorbitol | 5 |
| Cocoamidopropyl Betaine | 1 |
| Calcium Carbonate | 3 |
| Sodium Bicarbonate | 1 |
| Organic Stevia | 1 |
| Sea Salt | 2 |
| Zemea ® propanediol | 2 |
| Grapefruit Seed Extract | 0.8 |
| Peppermint oil | 0.3 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl Salicylate | 0.1 |
| Sorbitan monolaurate | 0.5 |
| Capryl caprylil Glucoside | 0.5 |
| Thymol | 0.1 |
| Menthol | 0.3 |
| Benzyl alcohol | 1 |
| Peppermint Flavor | 1.5 |
| Licorice (Root) Powder | 0.2 |
| Lemon Peel Powder | 0.2 |
| Cardamom Ext | 0.2 |
| Gum Arabic | 0.3 |
| White Oak Bark | 0.2 |
| Total | 100 |

TABLE 27

| | TP-LN-5B |
|---|---|
| Ingredient | % w/w |
| Water | 22.3 |
| Xylitol | 15 |
| Hydrated Silica | 15 |
| Xanthan Gum | 1 |
| Glycerin | 25 |
| Sorbitol | 5 |
| Cocoamidopropyl Betaine | 1 |
| Calcium Carbonate | 3 |
| Sodium Bicarbonate | 1 |
| Organic Stevia | 1 |
| Sea Salt | 2 |
| Zemea ® propanediol | 2 |
| Grapefruit Seed Extract | 0.8 |
| Peppermint oil | 0.3 |
| Spearmint oil | 0.1 |
| Benzoic acid | 0.1 |
| Sodium Benzoate | 0.1 |
| Zinc Salicylate | 0.1 |
| Pomegranate extract | 0.5 |
| Clove bud oil | 0.1 |
| Eucalyptol | 0.1 |
| Methyl Salicylate | 0.1 |
| Sorbitan monolaurate | 0.5 |
| Capryl caprylil Glucoside | 0.5 |
| Thymol | 0.1 |
| Menthol | 0.3 |
| Benzyl alcohol | 1 |
| Peppermint Flavor | 1.5 |
| Cardamom Ext | 0.2 |
| Gum Arabic | 0.3 |
| Total | 100 |

TABLE 28
Natural toothpaste

| | TP-B-1-A |
|---|---|
| Ingredient | % w/w |
| Water | 16.67 |
| Arrow Root | 2.28 |
| Calcium carbonate | 31.98 |
| Xylitol | 21 |
| Baking Soda | 10 |
| Xanthan gum | 0.76 |
| Water | 2 |
| Zemea ® propanediol | 2 |
| Grapefruit Seed Extract | 0.5 |
| Peppermint oil | 0.5 |
| Spearmint oil | 0.2 |
| Pomegranate extract | 0.3 |
| Clove bud oil | 0.1 |
| Eucalyptol | 0.1 |
| Methyl Salicylate | 0.1 |
| Sorbitan monolaurate | 0.5 |
| Plantasol ® caprylyl capryl glucoside | 0.5 |
| Thymol | 0.2 |
| Menthol | 0.3 |
| Benzyl alcohol | 1 |
| Cocoyl Glutamate | 5 |
| Stevia | 4 |
| Total | 100 |

TABLE 29
Natural toothpaste

| | TP-B-1-A2 |
|---|---|
| Ingredient | % w/w |
| Water | 15.77 |
| Arrow Root | 2.28 |
| Calcium carbonate | 31.98 |
| Xylitol | 21 |
| Baking Soda | 10 |
| Xanthan gum | 0.76 |
| Water | 2 |
| Zemea | 2 |
| Grapefruit Seed Extract | 0.5 |
| Peppermint oil | 0.25 |
| Spearmint oil | 0.1 |
| Pomegranate extract | 0.3 |
| Clove bud oil | 0.1 |
| Eucalyptol | 0.1 |
| Methyl Salicylate | 0.1 |
| Sorbitan monolaurate | 0.5 |
| Plantasol ® caprylyl capryl glucoside | 0.5 |
| Thymol | 0.2 |
| Menthol | 0.3 |
| Benzyl alcohol | 1 |
| Cardamom | 0.1 |
| Cocoyl Glutamate | 6 |
| Peppermint Flavor | 0.15 |
| Stevia | 4 |
| Total | 100 |

A toothpaste base was prepared, including the ingredients set forth below in Table 30:

TABLE 30
Toothpaste Base (TB-1)

Purified Water
Xylitol
Zeodent ® (Silica)
Sorbitol
Lauryl Glycoside
Sodium Methyl Cocoyl Taurate
Titanium dioxide
Stevia
Sodium Benzoate
Natural Flavor

Other toothpaste bases were prepared as shown in the following Tables 31 to 37, below:

TABLE 31

| Toothpaste Base (TB-2) | % w/w |
|---|---|
| Purified Water | 10-35 |
| Arrow Root | 0.01-10 |
| Xanthan Gum | 0.1-5 |
| Xylitol | 5-25 |
| Calcium Carbonate | 0.01-35 |
| Zeodent ® (Silica) | 0.01-35 |
| Sorbitol | 0.1-10 |
| Sodium Cocyl Glutamate | 0.1-10 |
| Lauryl Glycoside | 0.1-5 |
| Sodium Methyl Cocoyl Taurate | 0.1-5 |
| Titanium dioxide | 0.1-5 |
| Stevia | 0.01-2 |
| Sodium Benzoate | 0.01-2 |
| Natural Flavor | 0.1-5 |

TABLE 32

| Toothpaste Base (TB-3) | % w/w |
|---|---|
| Purified Water | 41 |
| Arrow Root | 2.6 |
| Xylitol | 18 |
| Calcium Carbonate | 25 |
| Sorbitol | 4 |
| Cocoyl Glutamate | 6 |
| Erythritol | 1 |
| Stevia | 0.25 |
| Sodium Benzoate | 0.15 |
| Natural Flavor | 2 |

TABLE 33

| Toothpaste Base (TB-4) | % w/w |
|---|---|
| Purified Water | 42 |
| Zeodent ® dental silica | 23 |
| Xylitol | 18 |
| Carboxymethyl cellulose | 0.5 |
| Sorbitol | 4 |
| Cocoyl Glutamate | 6 |
| Stevia | 0.25 |
| Sodium Benzoate | 0.15 |
| Xanthan Gum | 0.5 |
| Sodium bicarbonate | 3.6 |
| Natural Flavor | 2 |

TABLE 34
Toothpaste TP-B10-C

| Ingredient | Wt % |
|---|---|
| Thymol | 0.15 |
| Menthol | 0.15 |
| Benzoic Acid | 0.2 |
| Zemea ® propanediol | 3 |
| Grapefruit Seed Extract | 0.5 |
| Peppermint oil | 0.1 |
| Pomegranate extract | 0.3 |
| Sorbitan monolaurate | 0.2 |
| Plantasol ® caprylyl capryl glucoside | 0.2 |
| Benzyl Alcohol | 1 |
| Glycerin | 18 |
| Carboxymethyl cellulose | 0.2 |
| Xanthan Gum | 0.5 |
| Clove oil | 0.2 |
| Clove Flavor | 0.1 |
| Toothpaste Base (TB-1) | 75.2 |
| Total | 100 |

TABLE 35
Toothpaste TP-B10-C

| Ingredient | Wt % |
|---|---|
| Thymol | 0.15 |
| Menthol | 0.15 |
| Benzoic Acid | 0.2 |
| Zemea ® propanediol | 3 |
| Grapefruit Seed Extract | 0.5 |
| Spearmint oil | 0.1 |
| Pomegranate extract | 0.3 |
| Sorbitan monolaurate | 0.2 |
| Plantasol ® caprylyl capryl glucoside | 0.2 |
| Benzyl Alcohol | 1 |
| Glycerin | 18 |
| Carboxymethyl cellulose | 0.2 |
| Xanthan Gum | 0.5 |
| Clove oil | 0.2 |
| Clove Flavor | 0.1 |
| Toothpaste Base (TB-1) | 75.2 |
| Total | 100 |

TABLE 36
Toothpaste TP-B10-E

| Ingredient | Wt % |
|---|---|
| Thymol | 0.15 |
| Menthol | 0.15 |
| Benzoic Acid | 0.2 |
| Zemea | 3 |
| Grapefruit Seed Extract | 0.5 |
| Peppermint oil | 0.1 |
| Pomegranate extract | 0.3 |
| Sorbitan monolaurate | 0.2 |
| Plantasol ® caprylyl capryl glucoside | 0.2 |
| Eucalyptus oil | 0.5 |
| Glycerin | 18 |
| Carboxymethyl cellulose | 0.2 |
| Xanthan Gum | 0.5 |
| Clove oil | 0.2 |
| Clove Flavor | 0.1 |
| Toothpaste Base (TB-1) | 75.7 |
| Total | 100 |

TABLE 37
Toothpaste TP-B10-F

| Ingredient | Wt % |
|---|---|
| Thymol | 0.15 |
| Menthol | 0.15 |
| Benzoic Acid | 0.2 |
| Zemea | 3 |
| Grapefruit Seed Extract | 0.5 |
| Peppermint oil | 0.1 |
| Pomegranate extract | 0.3 |
| Sorbitan monolaurate | 0.2 |
| Plantasol ® caprylyl capryl glucoside | 0.2 |
| Eucalyptus oil | 0.5 |
| Benzyl alcohol | 0.5 |
| Glycerin | 18 |
| Carboxymethyl cellulose | 0.2 |
| Xanthan Gum | 0.5 |
| Clove oil | 0.2 |
| Clove Flavor | 0.1 |
| Toothpaste Base (TB-1) | 75.2 |
| Total | 100 |

### EXAMPLE 9

The toothpaste B formulations were tested using zone of inhibition study against S. aureus. An agar plate was inoculated with 0.3 mL of 10⁸ CFU/mL S. aureus and allowed to dry for 10 minutes. Afterwards, wells were prepared using a cork borer and 0.1mL of test toothpaste as well as control (TOMS of Maine) was introduced into the agar plate and incubated overnight. The zone of inhibition was measured.

Results are shown in Table 38 below.

### EXAMPLE 9A

The toothpaste B10 formulations were tested using zone of inhibition study against S. aureus and S. mutans Rapid kill (1 minute) test tube method described in ASTM-E2783-1198751-1 (suspension test).

10⁸ CFU mL⁻¹ microbial culture were prepared in a TSA media in order to determine the antimicrobial activity of toothpaste groups compared. All toothpaste samples were diluted 1:1 to reduce thickness. 0.9 mL diluted toothpaste was kept in contact with 0.1 mL microbial cultures for 1 minute and subsequently neutralized using drug inactivating solution (DE).

After serial dilution with DE, 0.5 mL dilutions were plated on Trypticase agar plates, incubated at 37° C. for 24 to 48 hours. Colony counts were determined and the log reduction of counts from control sample was calculated. Results are shown in Table 39 below.

### EXAMPLE 9B

In various embodiments, the compositions herein can be used as a botanical blend additive which can enhance the antimicrobial efficacy of other commercially available or products in development, in the fields comprising of but not limited to toothpaste, mints, chewing gum, mouth freshener.

An exemplary formulation of an antimicrobial blend was made as shown below in Table 40 (as a blend additive, such an antimicrobial blend was, in this embodiment, added as 6% of the overall oral care composition, with the base making up the remaining 94%):

TABLE 40
Antimicrobial Blend

| Ingredient | Wt % |
|---|---|
| Thymol | 0.15 |
| Menthol | 0.15 |
| Benzoic Acid | 0.2 |
| Zemea ® propanediol | 3 |
| Grapefruit Seed Extract | 0.5 |
| Peppermint oil | 0.1 |
| Pomegranate extract | 0.3 |
| Sorbitan monolaurate | 0.2 |
| Plantasol ® caprylyl capryl glucoside | 0.2 |
| Benzyl alcohol | 1 |
| Clove oil | 0.2 |
| Total | 6 |

### EXAMPLE 9C

In various embodiments, the antimicrobial blend was added to commercially available toothpaste and test for efficacy. Rapid kill test was carried our as follows. Rapid kill (1 minute) test tube method described in ASTM-E2783-1198751-1 (suspension test). 108 CFU mL⁻¹ microbial culture (S. mutans) were prepared in a TSA media in order to determine the antimicrobial activity of toothpaste groups compared.

All toothpaste samples were diluted 1:1 to reduce thickness. 0.9 mL diluted toothpaste was kept in contact with 0.1 mL microbial cultures for 1 minute and subsequently neutralized using drug inactivating solution (DE).

After serial dilution with DE, 0.5 mL dilutions were plated on Trypticase agar plates, incubated at 37° C. for 24 to 48 hours. Colony counts were determined and the log reduction of counts from control sample was calculated.

PBS was used as a control. In this study, an oral care product is considered effective, if the log10 reduction exhibits 3.0 or higher.

Results are shown below in Table 41:

### EXAMPLE 10

In various embodiments, the compositions herein can be used to prepare completely botanical oral spray, oral patch or the like. For oral patch, an oral composition containing Chitosan and other control release polymers can be blended together to obtain a composition that is viscous yet has slow release properties.

In certain embodiments, the compositions herein can comprise a hydrophilic polymer, a hydrophobic polymer, a hydrophobic-hydrophilic polymer; or a combination of a hydrophilic polymer and a hydrophobic-hydrophilic polymer.

For oral patch, the following example formulations can be used.

| Ingredient | % w/w |
|---|---|
| Sangelose (1%) | 30-40 |
| Chitosan (3%) | 30-50 |
| Tween 80 | 1-3 |
| Caprylyl Capryl Glucoside | 3-5 |
| Glycerin | 0.5-5 |
| Lactic Acid | 0.1-0.3 |
| 1,3 propanediol | 2-20 |
| Grapefruit Seed Extract | 0.5-2 |
| Peppermint oil | 0.03-1 |
| Spearmint oil | 0.03-1 |
| Benzoic acid | 0.1-0.5 |
| Sodium Benzoate | 0.1-0.5 |
| Zinc Salicylate | 0-0.5 |
| Pomegranate extract | 0.1-1 |
| Clove bud oil | 0.05-1 |
| Eucalyptol | 0.05-1 |
| Methyl Salicylate | 0.01-1 |
| Sorbitan monolaurate | 0.2-1 |
| Caprylyl capryl glucoside | 0.2-1 |
| Thymol | 0.05-0.2 |
| Menthol | 0.05-0.2 |
| Benzyl alcohol | 0.01-2 |
| Water | q.s |

For oral spray, the following example formulations can be used.

Alcohol free botanical oral spray (Range of ingredients)

| Ingredient | % w/w |
|---|---|
| Thymol | 0.01-0.1 |
| Menthol | 0.01-0.1 |
| Pomegranate extract | 0.05-0.3 |
| Grapefruit seed extract | 0.05-0.3 |
| Licorice extract | 2-20 |
| Cardamom oil or extract | 0.5-5 |
| Clove oil or extract | 0.5-10 |
| Peppermint extract | 1-5 |
| Spearmint extract | 1-5 |
| Ginger extract | 0.1-5 |
| Glycerin | 5-15 |
| Xylitol | 1-10 |
| Water | q.s |

Alcohol free botanical oral spray example formulations

### BOS-1

| Ingredient | % w/w |
|---|---|
| Thymol | 0.02 |
| Menthol | 0.03 |
| Pomegranate extract | 0.1 |
| Grapefruit seed extract | 0.1 |
| Licorice extract | 2 |
| Clove extract | 1 |
| Cardamom extract | 1 |
| Peppermint extract | 1 |
| Spearmint extract | 1 |
| Ginger extract | 0.2 |
| Glycerin | 10 |
| Xylitol | 10 |
| Water | q.s. |

The formulation can be used directly, or 1/10 or 1/20 dilution with water.

Alcohol free botanical oral spray BOS-2

| Ingredient | % w/w |
|---|---|
| Thymol | 0.01 |
| Menthol | 0.015 |
| Pomegranate extract | 0.05 |
| Grapefruit seed extract | 0.05 |
| Licorice extract | 2 |
| Clove bud extract | 0.5 |
| Cardamom extract | 0.5 |
| Peppermint extract | 0.5 |
| Spearmint extract | 0.5 |
| Ginger extract | 0.1 |
| Glycerin | 5 |
| Xylitol | 5 |
| Water | 85.75 |

Alcohol free botanical oral spray BOS-3

| Ingredient | % w/w |
|---|---|
| Thymol | 0.01 |
| Menthol | 0.03 |
| Pomegranate extract | 0.1 |
| Grapefruit seed extract | 0.1 |
| Licorice extract | 2 |
| Clove bud extract | 0.5 |
| Cardamom extract | 0.25 |
| Peppermint extract | 0.5 |
| Spearmint extract | 0.3 |
| Ginger extract | 0.2 |
| Glycerin | 5 |
| Xylitol | 10 |
| Water | 81 |

Alcohol free botanical oral spray BOS-4

| Ingredient | % w/w |
|---|---|
| Thymol | 0.01 |
| Menthol | 0.03 |
| Pomegranate extract | 0.1 |
| Grapefruit seed extract | 0.1 |
| Licorice extract | 2 |
| Clove bud extract | 0.5 |
| Cardamom extract | 0.1 |
| Peppermint extract | 0.5 |
| Spearmint extract | 0.3 |
| Ginger extract | 0.2 |
| Glycerin | 5 |
| Xylitol | 10 |
| Water | 81.15 |

Although the present technology has been described in relation to embodiments thereof, these embodiments and examples are merely exemplary and not intended to be limiting. Many other variations and modifications and other uses will become apparent to those skilled in the art. The present technology should, therefore, not be limited by the specific disclosure herein, and can be embodied in other forms not explicitly described here, without departing from the spirit thereof.

We claim:

1. An alcohol-free botanical oral care composition that achieves 3 or more logio reduction from control bacterial counts within 20 seconds of contact, comprising:
(a) a combination of 0.1 to 0.5% w/w pomegranate extract/oil and 0.3 to 1% w/w grapefruit seed extract;
(b) 0.05 to 0.08% w/w thymol, or 0.05 to 0.08% w/w menthol, or a combination thereof;
(c) 2 to 5% w/w 1,3 propanediol, or 5 to 15% w/w glycerin, or a combination thereof;
(d) 0.03 to 0.06% w/w peppermint oil, or 0.03 to 0.06% w/w spearmint oil, or a combination thereof;
(e) 0.3 to 0.8% w/w caprylyl capryl glucoside solubilizer; and
(f) 0.3 to 0.8% w/w one or more of organic surfactants selected from the group consisting of sorbitan mono laurate, sorbitan mono oleate, and sorbitan oleate decylglucoside;
wherein the ratio of solubilizer to surfactant is in the range of 1:1 to 1:20; and
wherein the combined concentration of solubilizer and surfactant does not exceed 2% of the composition.

2. The composition of claim 1, further comprising 0.1 to 0.2% w/w benzoic acid, 0.1 to

0. 2% w/w sodium benzoate, or a combination thereof

3. The composition of claim 1, further comprising one or more of an anti-irritant or anti-inflammatory agent.

4. The composition of claim 3, wherein the one or more anti-irritant or anti-inflammatory agent is calendula extract, methyl salicylate, zinc salicylate, or aloe gel extract.

5. The composition of claim 1, further comprising xylitol, erythritol, sorbitol, or a combination thereof.

6. The composition of claim 1, further comprising chlorhexidine gluconate.

7. The composition of claim 1, further comprising a natural colorant, wherein the natural colorant is a copper salt, chlorophyllin, a botanical or a combination thereof.

8. A consumer product comprising a composition of claim 1, in the form of an oral rinse, a toothpaste, an oral patch, a dental floss, or a lozenge.

9. The composition of claim 1, further comprising a mixture of:
(a) a hydrophilic polymer; and
(b) a hydrophobic-hydrophilic polymer.

10. A method of treating a wound, the method comprising contacting the wound with a composition of claim 1.

11. A medical device coated with the composition of claim 1.

12. An alcohol-free natural toothpaste composition that achieves more than 15 mm zone of inhibition against S. aureus comprising:
a) a combination of 0.1 to 1% w/w pomegranate extract/oil and 0.5 to 2% w/w grapefruit seed extract;
b) 0.05 to 1% w/w thymol, or 0.05 to 1% w/w menthol, or a combination thereof;
c) 2 to 20% w/w 1,3 propanediol, or 8 to 50% w/w glycerin, or a combination thereof; and
d) 0.03 to 1% w/w peppermint oil, or 0.03 to 1% w/w spearmint oil, or a combination thereof; and
e) 0.01 to 3% w/w clove oil, or clove extract, or a combination thereof

13. The toothpaste composition of claim 12, further comprising one or more of the following:
f) 0.2 to 1% w/w caprylyl capryl glucoside solubilizer; or
g) 0.2 to 1% w/w one or more of organic surfactants selected from the group consisting of sorbitan mono laurate, sorbitan mono oleate, cocoamidopropyl betaine, bio-Saponin, sodium cocoyl glutamate, and sorbitan oleate decylglucoside; or
h) 0.05 to 1% w/w eucalyptol; or 0.01 to 2% w/w benzyl alcohol, or a combination thereof; or
i) 0.01 to 20% w/w texturing agent wherein the texturing agent is silica, sodium methyl cocoyl taurate, carboxymethyl cellulose titanium dioxide, or combination thereof.

14. The composition of claim 12, further comprising 0.1 to 0.5% w/w benzoic acid, or 0.1 to 0.5% w/w sodium benzoate, or a combination thereof.

15. The composition of claim 12, further comprising 0.01 to 5% w/w carrageenan, or 0.01 to 1% w/w methyl cellulose, or 0.1 to 2% w/w xanthan gum, or 0.5 to 20% w/w coconut oil thickener, or any combination thereof.

16. The composition of claim 12, further comprising a natural colorant, wherein the natural colorant is a copper salt, chlorophyllin, a botanical extract or a combination thereof.

17. The composition of claim 12, further comprising one or more of an anti-irritant or anti-inflammatory agent.

18. The composition of claim 17, wherein the one or more anti-irritant or anti-inflammatory agent is calendula extract, methyl salicylate, zinc salicylate, and aloe gel extract.

19. The composition of claim 12, further comprising xylitol, erythritol, sorbitol or a combination thereof.

20. The composition of claim 12, further comprising one or more of the following flavoring agents: clove extract or powder, clove bud oil, clove oil, cardamom extract or powder, peppermint extract, spearmint extract, licorice root extract or powder, white oak bark extract or powder, gotu kola extract or powder, cinnamon extract or powder, lemon extract or powder, lemon peel extract or powder, or orange extract or powder.

21. The composition of claim 20, wherein the one or more flavoring agents is present in an amount of 0.05-5% w/w extract or 0.1 to 0.5% w/w powder.

22. The composition of claim 12, further comprising 0.1 to 1% w/w of the marbling agent acacia Arabica powder.

23. An oral care composition comprising 5 to 10% w/w of the composition of claim 12, wherein the oral care composition exhibits 3 or more logio reduction from control bacterial counts within 30 seconds of contact.

24. An alcohol free oral spray comprising: 0.01 to 0.1% w/w thymol; 0.01 to 0.1% menthol; 0.05 to 0.3% w/w pomegranate extract; 0.05 to 0.3% grapefruit seed extract; 1 to 5% peppermint oil or peppermint extract; 1 to 5% w/w spearmint oil or extract; 2 to 20% licorice extract; 0.5 to 10% clove oil or extract; 0.5 to 5% w/w cardamom oil or extract, 5 to 15% w/w glycerin, 1 to 10% xylitol; 0.1 to 0.5% w/w ginger extract and water.

25. The composition of claim 24, further comprising a natural colorant, wherein the natural colorant is a copper salt, chlorophyllin, a botanical extract or a combination thereof

26. The composition of claim 24, further comprising one or more of the following: nutmeg oil or extract; mace oil or extract; camphor powder or oil; curcumin oil or extract; and curcumin extract or stevia extract.
